(19) United States
(12) Patent Application Publication
(10) Pub. No.: US 2012/0301940 A1
(43) Pub. Date: Nov. 29, 2012

(54) METHOD FOR PRETREATING CRUDE OIL USING MICROORGANISM

(76) Inventors: Ahmad Ramli Bin Mohd Yahya, Palau Pinang (MY); Mohamad Nasir Bin Mohamad, Palau Pinang (MY); Nur Asshifa Binti Md Noh, Palau Pinang (MY)

(21) Appl. No.: 13/520,764

(22) PCT Filed: Mar. 31, 2010
(86) PCT No.: PCT/MY2010/000047

§ 371 Date: Aug. 9, 2012

(30) Foreign Application Priority Data
Dec. 17, 2009 (MY) PI 20095437

(51) Int. Cl. C12P 5/00

(52) U.S. Cl. 435/166

(57) ABSTRACT

A method for pretreating a crude oil prior to a crude oil distillation process comprising the step of propagating a culture of a hydrocarbon-utilizing and biosurfactant-producing microbial strain and treating the crude oil with the propagated culture.

### FIELD OF INVENTION

The present invention relates to an improvement process in the crude oil refinery industry. In more particular, the present invention relates to a method for pretreating hydrocarbon using a hydrocarbon-utilizing and biosurfactant-producing microorganism to facilitate the crude oil distillation process.

### BACKGROUND OF THE INVENTION

Distillation of crude oil is a common method for the fractionation of petroleum producing desirable products, such as automotive fuel and kerosene. In the early days of the refining industry, distillation was recognized as a way to produce a desirable product, such as kerosene as a lamp oil. Distillation became the process of choice for petroleum refining, in which the most common petroleum fractions including petrol, kerosene, diesel, and lube oil can be obtained therefrom.

Conventionally, the distillation process is conducted without any involvement of biological materials. This current practice for decades has been able to produce distillate of interest with standard quality. The process thus involves tremendous consumption of thermal energy and requires a long period of time.

There are a few patented technologies disclosed in the prior arts relating to a recovery method or distillation method for hydrocarbon or crude oil. U.S. Pat. No. 7,172,686 relates to methods for separating components of a mixture, such as crude oil. The methods disclosed is capable of increasing the yield of individual components while decreasing the yield of residue. However, this invention focuses mainly on the system of the crude oil distillation and the use of separation column in the system to obtain the desired yield of products but does not shorten the distillation period.

Another U.S. Pat. No. 6,413,415 relates to a method for high-temperature short-time distillation of residual oils. This method comprises feeding the oil to a mixer with granular hot coke, which serves as a thermal transfer medium. However, it is used for treating residual oils originating from crude oil refining, natural bitumen and/or tar sands, instead of being used for pretreating the crude oil prior to distillation.

Most of the distillation processes are not based on biochemical approaches. In the Canadian Patent No. CA2260576, biosurfactant produced by the microorganism such as Pseudomonas spp. is of high viscosity. However, the microorganisms are merely disclosed for use in preparing emulsion of oil.

There is also use of microbial consortium in a process for enhanced recovery of crude oil from oil wells as disclosed in U.S. Patent No. 2007092930. The microbial consortium contains three hyperthermophilic, barophilic, acidodenic and anaerobic bacterial strains, which is capable of producing a variety of metabolic products which are capable of increasing sweep efficiency of crude oil from oil bearing poles of rock formation. The metabolic products include carbon dioxide, methane, biosurfactant, volatile fatty acids and alcohols in the presence of specially designed nutrient medium. However, the microbial strains required are in a wide variety.

There is no prior art disclosing a process for distilling crude oil using microorganism, such asPseudomonas spp., which is capable of utilizing hydrocarbon and producing a biosurfactant for use in the distillation process. It is therefore desirable for the present invention to provide a process for distilling crude oil which is capable of increasing the volume of distillate with comparable quality as well as reducing time, cost and energy incurred.

### SUMMARY OF INVENTION

The primary object of the present invention is to provide a pretreatment process to improve crude oil or hydrocarbon distillation process by using a hydrocarbon-utilizing and biosurfactant-producing microorganism.

Another object of the present invention is to develop a method for treating crude oil or hydrocarbon with a hydrocarbon-utilizing and biosurfactant-producing microorganism prior to crude oil distillation process.

Still another object of the present invention is to provide a method for pretreating crude oil or hydrocarbon prior to the distillation process which is capable of shortening distillation time by shortening the period to obtain liquid distillate or by lowering the temperature where distillate can be obtained earlier.

Yet another object of the present invention is to provide a method for pretreating crude oil or hydrocarbon prior to the distillation process which is capable of obtaining an increasing volume of the distillate yet maintaining the quality and energy/calorie value of the distillate.

At least one of the preceding objects is met, in whole or in part, by the present invention, in which one of the embodiments of the present invention describes a method for pretreating a crude oil prior to a crude oil distillation process comprising the step of propagating a culture of a hydrocarbon-utilizing and biosurfactant-producing microbial strain and treating the crude oil with the propagated culture.

Another embodiment of the present invention discloses that the hydrocarbon-utilizing and biosurfactant-producing microbial strain is Pseudomonas spp. Preferably, the microbial strain is Pseudomonas aeruginosa.

Still another embodiment of the present invention discloses that the biosurfactant produced is rhamnolipid. Rhamnolipid (a glycolipid biosurfactants) is specifically produced by Pseudomonas aeruginosa. Other types of biosurfactants produced by bacteria from the genera of Pseudomonas are fatty acids and lipopeptides.

Another embodiment of the present invention discloses that the biosurfactant produced in the propagated culture is 1 g/L to 15 g/L. Preferably, approximately 1 g/L to 3 g/L of the biosurfactant is used for pre-treating 50 mL of crude oil.

The biosurfactant produced by the hydrocarbon-utilizing cultured microbial strain has an ability to provide a 2 to 3-fold decrease of distillation times without affecting or reducing the quality of the distillate collected from the crude oil distillation process. The ability of the cultured microbial strain in producing effective biosurfactant for acceleration of the emulsification or biodegradation process is not disclosed in any of the prior methods of conventional distillation.

One skilled in the art will readily appreciate that the present invention is well adapted to carry out the objects and obtain the ends and advantages mentioned, as well as those inherent therein. The embodiments described herein are not intended as limitations on the scope of the invention.

### BRIEF DESCRIPTION OF THE DRAWINGS

For the purpose of facilitating an understanding of the invention, there is illustrated in the accompanying drawing the preferred embodiments from an inspection of which when considered in connection with the following description, the invention, its construction and operation and many of its advantages would be readily understood and appreciated.

FIG. 1 shows the same chromatogram detected for distillate of control (804) and treated crude oil Tukau TK 57S (803) as described in one of the preferred embodiments of the present invention.

FIG. 2 shows the same chromatogram detected for distillate of control (801) and treated crude oil Angsi (802) as described in one of the preferred embodiments of the present invention.

### DETAILED DESCRIPTION OF THE INVENTION

The present invention relates to an improvement process in the crude oil refinery industry. In more particular, the present invention relates to a method for pretreating hydrocarbon using a hydrocarbon-utilizing and biosurfactant-producing microorganism to facilitate the crude oil distillation process.

Hereinafter, the invention shall be described according to the preferred embodiments of the present invention and by referring to the accompanying description and drawings. However, it is to be understood that limiting the description to the preferred embodiments of the invention and to the drawings is merely to facilitate discussion of the present invention and it is envisioned that those skilled in the art may devise various modifications without departing from the scope of the appended claim.

The present invention discloses a method for pretreating a crude oil prior to a crude oil distillation process comprising the step of propagating a culture of a hydrocarbon-utilizing and biosurfactant-producing microbial strain and treating the crude oil with the propagated culture. Another embodiment of the present invention discloses that the hydrocarbon-utilizing and biosurfactant-producing cultured microbial strain is Pseudomonas spp., most preferably P aeruginosa. P. aeruginosa is selected as the most preferable microbial strain to be applied in the present invention because it is identified as a good hydrocarbon-utilizing bacteria as well as a good biosurfactant producer. From the potential shown by this microorganism, it plays an important role to improve the existing conventional refinery and distillation process in hydrocarbon refinery industry. Reducing the distillation times translates to energy and cost savings in producing petroleum products.

Still another embodiment of the present invention discloses that the biosurfactant produced is rhamnolipid. Rhamnolipid (a glycolipid biosurfactants) is specifically produced by Pseudomonas aeruginosa. Other types of biosurfactants produced by bacteria from the genera of Pseudomonas are fatty acids and lipopeptides. The ability of the P. aeruginosa culture to produce such emulsifying surfactant compounds whether extracellularly or at the surface of the cell membrane is one of the determining characteristics of its ability to influence crude oil rheological and physicochemical properties.

The rhamnolipid produced by P. aeruginosa culture is capable of facilitating the distillation process without affecting or reducing the quality of distillate obtained from the crude oil. It has an ability to emulsify insoluble compound, such as oil, resulting in emulsification and slight degradation of the compounds. Rhamnolipid reduces oil viscosity by breaking down the molecular structure of crude oil, making it more fluidal. In addition, it plays major roles in the enhancing performance of distillation by the emulsification of hydrocarbon chains in crude oil. This effectively loosens hydrocarbon compounds trapped within the complex hydrocarbon chains. Consequently, this phenomenon results in shorter distillation time as well as lower and wider temperature range to obtain distillate. It also results in the increase in distillate volumes obtained. The ability to collect distillate at lower temperatures compared to that of the control, shortens the distillation times. Microbial activity, via the production of effective biosurfactant, is deemed the main factor that enhances crude oil distillation performance.

According to the preferred embodiment of the present invention, the culture of P. aeruginosa can be propagated in shake flask or fermenter. It is because emulsification by biosurfactants is a cell density dependent phenomenon, that is, the greater the number of cells, the higher the concentration of extracellular product. Therefore, in order to achieve an optimal production of rhamnolipid to be treated on the crude oil later, fermentation process is preferably conducted. Simultaneously production of biosurfactant and crude oil treatment is also possible. However, preliminary study recorded that this combined process may take a week before distillation can be conducted, depending on the type or physical properties of the crude oil and the concentration of biosurfactant (rhamnolipid) in the solution.

Accordingly, the culture broth or supernatant of the bacterial strain is introduced into crude oil at a range of ratio of 5% to 30% by volume of the crude oil. The concentration of the surfactant produced by the P. aeruginosa strain is in the range of 1 g/L to 15 g/L. And this concentration can be fixed at 1 to 3 g/L for every treatment of 50 mL crude oil. Either the spent culture or supernatant can be used as treatment on the crude oil, since the amount of rhamnolipid in these two liquids does not differ significantly. The mixture can be shaken at 100 rpm to 200 rpm for typically, 15 to 30 minutes or even until 24 hours to ensure complete mixing prior to distillation.

Subsequently, the mixture can be distilled to compare the performance between the crude oil which is treated with the improved microbial culture and the untreated crude oil. In accordance with the preferred embodiment of the present invention, the performance can be observed by comparing the distillation times. Distillation times are period when the distillation process starts (0 minute) until all distillate is collected and temperature does not increase any higher (when the timing is stopped). In accordance with the preferred embodiment of the present invention, the distillate analysis can be carried out by calorimetric test, gas chromatography-mass spectrometry (GC-MS) analysis, or both.

The comparison studies between these treated and untreated crude oils are further described in the examples. The results of these analysis are capable of demonstrating a lower distillation time as well as a higher volume of the distillate obtained. Treatment with the improved culture of P. aeruginosa is capable of providing a 2- to 3-fold decrease of distillation times without affecting or reducing the quality of the distillate collected. Distillate analyses by calorimetric test and GC-MS also show identical distillate quality when compared with distillations without pretreatment with the biological materials.

The present disclosure includes as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to without departing from the scope of the invention.

### EXAMPLE

Examples are provided below to illustrate different aspects and embodiments of the present invention. These examples are not intended in any way to limit the disclosed invention, which is limited only by the claims.

### Example 1

The improved culture of P. aeruginosa, namely USM-AR2, was inoculated into crude oil samples from Tukau and Angsi at 12% and shaken at 200 rpm for about 15 minutes to ensure mixing prior to distillation. The mixture was distilled to compare the performance between the crude oil which was treated with the improved microbial culture and the untreated crude oil. The performance was observed by comparing the distillation times. The experiments were carried out in duplicates and the distillate analysis was carried out by calorimetric test and GC-MS analysis. The results showed 2-3 fold decrease of distillation times with higher distillate volumes.

### Example 2

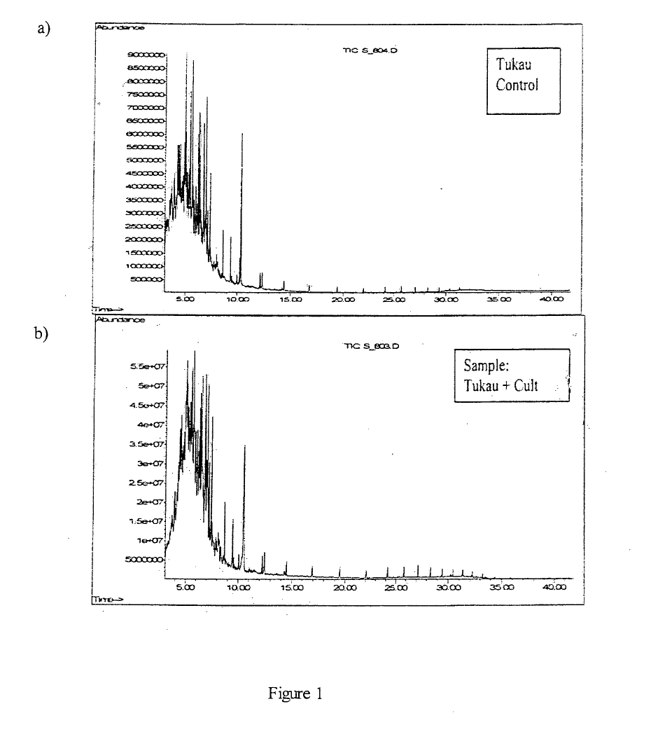

A comparison of distillation performance between treated and untreated crude oil Angsi with the USM-AR2 culture was conducted. The averaged results of duplicates were summarized and tabulated in Table 1. Accordingly, three separate treatments, including 5 ml of the USM-AR2 culture shaken for 15 min, 15 ml of culture shaken for 15 min and 5 ml of culture shaken for 12 hours, have been applied for the analysis. The results of this analysis show that the time and temperature for obtaining the distillate were reduced and the volume of the distillate obtained was increased. The same chromatogram detected for distillate of control (801) and treated crude oil Angsi (802) is illustrated in FIG. 1(a) and (b). GC-MS analysis showed no difference between chromatograms of the compounds detected in distillate from treated or untreated crude oil. Calorimetric tests showed paralleled results, where the calories value of the distillate obtained maintained high when treated with bacterial culture. The value between control and all treated samples showed no significant difference. These two factors confirmed that the quality of the distillate was not decreased. The degradation and emulsification process by the microbial culture to be under control as it does not degrade the oil excessively. The complexity of petroleum chains may cause the same compounds in distillate to come out at different temperature. This is shown in results where the same compounds were identified by GC-MS but collected at different temperature range.

TABLE 1

| | Control/ | |
|---|---|---|
| Parameters | Conventional way | Treated |
| Time taken for obtaining | 25 | 8 |
| distillate (min) | | |
| Temperature when | 200-210° C. | 100-200° C. |
| distillate obtained | | |
| Highest temperature reached | 215° C. (30 min) | 205° C. (20 min) |
| Volume of distillate (ml) | 0.5 | 10.0 |
| Calorie value (kcal/g) | 10.77 | 10.65 |
| GC-MS analysis | No difference | No difference |

### Example 3

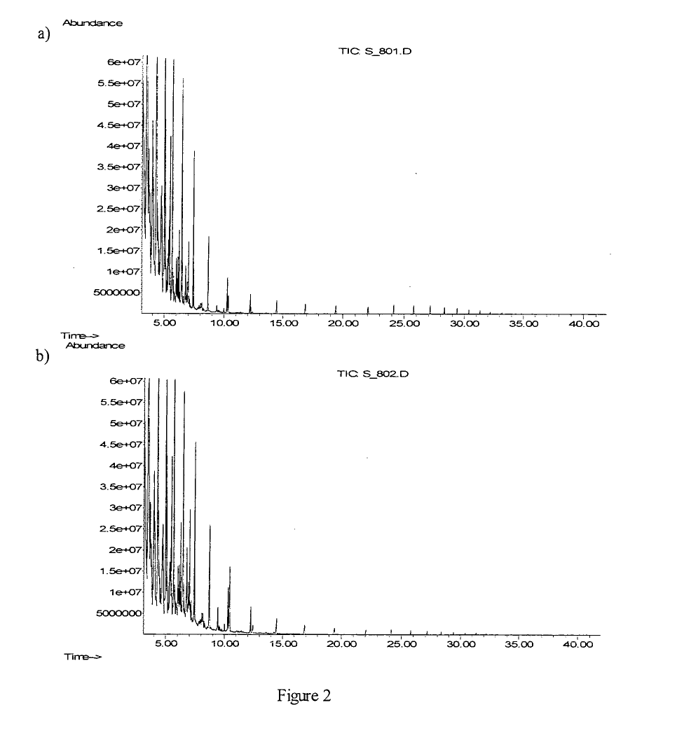

A comparison of distillation performance between treated and untreated crude oil Tukau TK-57S with the USM-AR2 culture was conducted, in which a 5 ml and a 15 ml of culture were prepared and shaken for 30 minutes. The averaged results of duplicates were summarized and tabulated in Table 2. As shown in Table 2, this result shows the effectiveness of the distillation process in which there were reductions in time and increase in volume of distillate. As shown in FIG. 2, there was no difference found in the GCMS analysis for both distillates of treated (803) and untreated crude oil (control) (804) as the same peaks representing the same components were identified in the chromatogram for both studies.

TABLE 2

| | Control/ | |
|---|---|---|
| Parameters | Conventional way | Treated |
| Time taken for obtaining | 22 | 10 |
| distillate (min) | | |
| Temperature when | 100-160° C. | 100-160° C. |
| distillate obtained | | |
| Highest temperature reached | 215° C. (20 min) | 215° C. (20 min) |
| Volume of distillate (ml) | 0.5 | 5.0 |
| Calorie value (kcal/g) | 11.87 | 12.05 |
| GC-MS analysis | No difference | No difference |

TABLE 3
Possible parameters for the pretreatment
of crude oil prior to distillation

| Parameters | Range | |
|---|---|---|
| Ratio of culture containing | 5~30% | |
| rhamnolipid to crude oil | | |
| Method of incubation of culture and | Shaken or unshaken (left on bench) | |
| crude oil | | |
| Time of mixing of culture and crude | 15 min~12 | hours |
| oil (if shaken) | | |
| Time of mixing of culture and crude | 1 hour~24 | hours |
| oil (if unshaken) | | |
| Agitation of mixture (if shaken) | 100~200 | rpm |
| Initial pH of culture before | pH 5~7 | |
| fermentation | | |
| Temperature during cultivation of | 25~30° | C. |
| USM-AR2 (P. aeruginosa) | | |
| Temperature during the treatment | 25~30° | C. |
| (mixing) of culture and crude oil | | |
| Broth for crude oil biotreatment | Culture or supernatant | |
| Concentration of rhamnolipid in | 2.5~3.5 | g/L |
| culture before treatment | | |

1-6. (canceled)

7. A method for pretreating a crude oil prior to a crude oil distillation process to reduce distillation time, comprising the steps of:
propagating a culture of a hydrocarbon-utilizing and biosurfactant-producing microbial strain; and
treating the crude oil with the propagated culture by breaking down the molecular structure of the crude oil;
wherein the biosurfactant produced in the propagated culture is present in the range of 1 g/L to 15 g/L.

8. The method according to claim 7, wherein the hydrocarbon-utilizing and biosurfactant-producing microbial strain is Pseudomonas spp.

9. The method according to claim 8, wherein the Pseudomonas spp. is Pseudomonas aeruginosa.

10. The method according to claim 7, wherein the biosurfactant produced is rhamnolipid.

11. The method according to claim 7, wherein 1 g/L to 3 g/L of the biosurfactant is used for pre-treating 50 mL of crude oil.
